(19) United States
(12) Patent Application Publication
(10) Pub. No.: US 2024/0164697 A1
(43) Pub. Date: May 23, 2024

(54) SYSTEM AND METHODS FOR DETECT AUTONOMIC DYSREFLEXIA DETECTION USING MACHINE LEARNING CLASSIFICATION

(71) Applicant: Purdue Research Foundation, West Lafayette, IN (US)

(72) Inventors: Brad S Duerstock, West Lafayette, IN (US); Shruthi Suresh, Overland Park, KS (US); Ana Karina Kirby, West Lafayette, IN (US); Thomas Everett, Indianapolis, IN (US); Sidharth Pancholi, West Lafayette, IN (US)

(73) Assignee: Purdue Research Foundation, West Lafayette, IN (US)

(21) Appl. No.: 18/232,789

(22) Filed: Aug. 10, 2023

Related U.S. Application Data
(60) Provisional application No. 63/396,888, filed on Aug. 10, 2022.

(51) Int. Cl. A61B 5/00; A61B 5/0205; A61B 5/024; A61B 5/0533; A61B 5/332; A61B 5/388

(52) U.S. Cl. CPC A61B 5/4035; A61B 5/02055; A61B 5/7267; A61B 5/7282; A61B 5/02438; A61B 5/0533; A61B 5/332; A61B 5/388

(57) ABSTRACT

A system for detecting autonomic dysreflexia (AD) may measure skin nerve activity (skNA), galvanic skin response (GSR), heart rate, and skin temperature of a subject. The system may extract, a plurality of features from the measurements. The features may include medianNN, average iskNA, number of bursts, RMSSD, pNN5, or a combination thereof. The system may classify, based on a machine learning model, the plurality of features to identify the onset of AD in the subject. The system may output, in response to the onset of AD, a message indicative of the onset of AD.

### CROSS REFERENCE TO RELATED APPLICATIONS

This application claims the benefit of U.S. Provisional Application No. 63/396,888 filed Aug. 10, 2022. the entirety of which is incorporated by reference herein.

### TECHNICAL FIELD

This disclosure relates to detection of Autonomic Dysreflexia (AD) and, in particular, to automated detection of AD using machine learning.

### BACKGROUND

Autonomic Dysreflexia (AD) is a unique manifestation in individuals with spinal cord injury (SCI) above the T6 (thoracic) level. AD is characterized clinically by an acute increase in systolic blood pressure (SBP) of at least 20 mmHg and sudden, uninhibited sympathetic discharges, and may be accompanied by bradycardia. It is a potentially life-threatening condition commonly initiated by irritation or noxious stimuli below the level of injury or triggers with 85% of cases of AD being triggered by urinary tract infections, impacted bowels. AD causes debilitating symptoms above the level of injury and can cause a dangerous increase in blood pressure (BP) if left untreated. In a study of life-threatening instances of AD, 22% of cases resulted in death. However, being familiar with the symptoms and triggers of AD remains the standard approach for managing AD for newly injured tetraplegics. Only 41% of persons with SCI or their family had heard of AD even though 22% of individuals with SCI reported symptoms consistent with unrecognized AD. Meticulous monitoring of telltale symptoms of AD can prevent the rapid escalation of AD-induced hypertension and reduce risks to personal health if managed quickly.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a first example of a system.

FIG. 2A-D illustrates plots of signals derived from an ECG signal via signal processing.

FIG. 3A-D illustrates correlations of various SKNA features utilized for the classification of AD and non-AD events, along with the feature distribution.

FIG. 4 illustrates an AD event that occurred during the performance of CRD.

FIG. 5 illustrates an example of a neural network architecture for detecting AD and Non-AD events.

FIG. 6 illustrates a second example of a system.

### DETAILED DESCRIPTION

Spinal cord injury (SCI) is a complex medical condition that often leads to various secondary complications, including autonomic dysreflexia (AD). Recognition of Autonomic Dysreflexia (AD) symptoms can take time and identifying the source of noxious stimuli occurring in paralyzed parts of the body may be difficult to the detriment of the individual, especially for those who are newly adapting to living with paralysis. A noninvasive, continuous monitoring device is needed to readily detect instances of AD particularly for asymptomatic or ‘silent’ AD episodes, which can be potentially harmful to cardiovascular end-organs due to recurrent episodes of paroxysmal rises in blood pressure without concomitant symptoms.

Clinically, medical professionals use blood pressure monitoring to diagnose AD. However, this method is impractical for continuous monitoring for the presence of AD for long-term use as it restricts individuals' activities and can be affected by movements such as wheeling or transferring. The tactile and sonorous stimuli to measure BP can be distracting, interrupting activities of daily living (ADLs) or sleep. Moreover, the sampling rate of measuring blood pressure is quite low, at most every five minutes, when using ambulatory blood pressure monitoring systems (ABPM). Interpretation of the ABPM data requires a trained clinician and typically a computer and data processing software as well as the required training on ABPM usage, data analysis and interpretation. This hinders widespread adoption of AD monitoring in the SCI community. According, a system and method for early detection of AD is provided. The system and methods described herein provide various technical advancements including a sensitive yet noninvasive method of detecting the onset of AD, which can be adopted easily into clinical practice and for at home use.

The development of AD in response to SCI has been investigated in human and animal models. Rat models in particular have been used by other researchers to study the onset of AD. These studies often focus on comparing pharmacological interventions when severe AD occurs as well as the mechanism of the onset of AD. However, few researchers have explored the unique physiological signatures of AD using a multimodal array of sensors that is not dependent upon a significant increase in blood pressure. These studies rely entirely on the pre-determined patterns in BP measured by a telemetry system to identify the onset of AD event induced by a trigger.

This disclosure demonstrates the use of multimodal, wearable sensor technologies to characterize the unique signatures of AD from different sympathetic stressors and the use of machine learning models to automate the process. Various experimentation on rodents were performs, though the technologies described herein are transferrable to human subjects. While various technical advancements are described or made evident herein, a key advantage of the system and methods described herein is the replacement of the need for using blood pressure measurements, which is the gold standard for detecting AD. Whereas blood pressure through a typical ambulatory BP cuff is fairly obtrusive and has low temporal resolution, the system and methods described herein provide higher resolution for feature extraction and fast detection of the onset of AD.

Time domain features are particularly advantageous as they can be directly calculated from the signals without requiring complex transformations. Various machine learning algorithms, including Decision Trees, Support Vector Machines (SVM), Linear Discriminant Analysis (LDA), K-Nearest Neighbors (KNN), and Random Forest (RF), have traditionally been employed to train and predict physiological signal episodes. However, Deep Neural Networks (DNN) have gained popularity due to their ability to outperform other algorithms and adapt to changing parameters. Neural networks have demonstrated remarkable effectiveness in disease diagnosis, especially in tasks such as disease classification and prediction. By leveraging pathological or physiological data, neural networks allocate greater weight to important features, enabling accurate categorization of diseases and insightful predictions. Through the training process, neural networks autonomously learn to assign higher significance to relevant aspects of the data, enhancing their understanding of the underlying patterns. Their ability to intelligently analyze diverse facets of the input enables neural networks to excel in identifying crucial patterns and providing precise classifications or predictions. As a result, they significantly contribute to effective disease diagnosis and drive advancements in healthcare outcomes.

The system and methods described herein provide an innovative approach for the early and continuous detection of AD in individuals with SCI. In various aspects, the system may utilize a machine learning model, such as a DNN architecture. for accurately detecting AD events using only the SKNA signal. In some experimentation, a DNN model was trained on labeled data sets and outperformed previous methods that utilized multiple physiological features, demonstrating that SKNA alone is sufficient for detecting AD-related changes in the autonomic nervous system. The model's performance was evaluated on 30% of unseen data, confirming its robustness and ability to generalize well. These findings suggest that the proposed DNN model has the potential for clinical applications in AD detection. Accordingly, SKNA measurement alone may provide a reduce complexity and cost and increase robustness compared to the multi-modal approach, though both approaches provide technical advances.

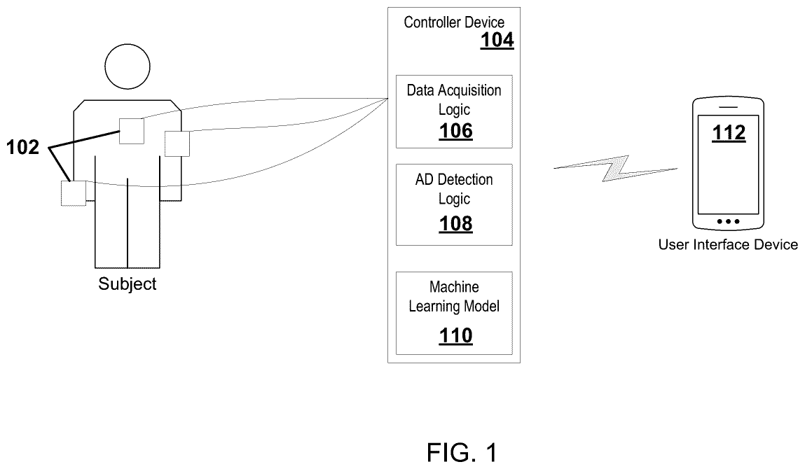

FIG. 1 illustrates a first examine of a system for AD detection. The system may include a plurality of sensors and controller device. The sensors may include non-invasive sensors 102 including, but not limited to, an electrocardiogram (ECG) sensor, a galvanic skin response (GSR) sensor, a heart rate sensor, and/or a skin temperature sensor. The system may further include a controller device 102 and the sensors may communicate with the controller device 104 through a wired or wireless connection and directly or indirectly via a network. Alternatively, the sensors may be included with the controller device 102 such that the sensor(s) and other processors, hardware, logic, etc. the controller device are an integrated device.

The controller device 102 may include various logic including data acquisition logic 106, AD detection logic 108, and a machine learning model 110.

The data acquisition logic 106 may perform signal processing on the signals received by the sensor to performs measurements. For example, the measurements may include ECG measurements, skin nerve activity (skNA), GSR, heart rate, and/or skin temperature of a subject.

The AD detection logic 108 may extract features based on the measurements. The features may include features representative of sympathetic activity, features of cardiovascular function, and feature(s) representative of other physiological functions, such as GSR and skin temperature. By way of example the extracted features may include medianNN, average iskNA, number of bursts, RMSSD, pNN5, or a combination thereof. Additional and alternative features and combinations of features are described herein may be advantageous depending on the approach.

The controller device may further include a machine learning model. The machine learning model is trained based on a plurality of training data comprising labeled features derived from time series data sensor data. Thus, the sensors may provide, for example, ECG data, skin nerve activity (skNA) data, blood pressure (BP) GSR data, and a skin temperature data, wherein features may be extracted and labeled for training purposes. In some examples, the machine learning model may include a neural network. The neural network may include an input and output layer with multiple hidden layers. These can be expanded to include a multilayer perceptron model or a convolutional neural network which can be trained using data collected.

The AD detection logic may access the machine learning model to perform classification of the features and detect the onset of AD in the subject.

The onset of AD as described herein means any physiological response has been shown to indicate that AD is occurring or is about to occur with or without symptoms. A technical advantage of the AD detection logic described herein as that the detection precedes the typical spike in blood pressure that historically has been used as the gold standard for clinically verifying the occurrence of AD.

In response to detection of the onset of AD, the AD detection logic may generate a message, or more generally, information representative of the onset of AD. The AD detection logic may cause the information to be output. Outputting the information may include, storing the information in a memory, communicating the information over a network, and/or causing the information to be displayed. In some examples, the system may include a user interface device 112 which is capable of displaying the information or notifying the subject (or some other person) of the onset of AD.

It should be appreciated that the data acquisition logic 106, AD detection logic 108, and/or machine learning model 110 may be distributed partially or completely upon numerous devices. For example, the controller device may only have the data question logic 106 whereas the AD detection logic 108 and/or machine learning model 110 may be in the user interface device 112 or in a cloud infrastructure in communication with the controller device 104 and/or the user interface device 112. Alternatively or in addition, the controller device 104 and user interface device 112 may be the same device and/or share hardware resources. The system 100 may be implemented in many ways with hardware and infrastructure not necessarily shown in FIG. 1.

### Examples and Experimentation for Feature Selection Techniques for a Machine Learning Model to Detect Autonomic Dysreflexia With Multi-Modal Parameters

Current healthcare practices revolve around human expert assessments of correlations between symptoms and diagnoses. Machine learning (ML) has been applied to various areas of healthcare and has enormous potential to improve detection of disease for rapid point-of-care treatment, help clinicians with making diagnostic decisions (decision support system)[ [symbol] , and improve individual management of chronic health conditions.

ML techniques can contribute to finding patterns and trends that contribute to the knowledge about different disease states as well as help diagnose them early. Supervised ML methods are among some of the most common approaches used in the clinical setting due to the large amount of annotated data which is available. Some applications of ML to healthcare settings include automated arrhythmia analysis tools using physiological data such as electrocardiogram (ECG) or alerts for low oxygen saturation using photoplethysmography (PPG). However, despite its strengths, ML cannot identify relationships that are not present in the data; therefore, data veracity is critical to any accurate ML model.

Feature extraction is the process of reducing a set of raw/preprocessed data into a smaller set of features which represent the key qualities of the data. In healthcare data extraction of relevant features is often guided by physiological understanding of the mammalian system. Feature selection prevents overfitting of a machine learning model to improve performance and provide faster, more cost-effective models. Through feature selection, the original representation of the features is not altered, and the original semantics are preserved. Additionally, through specific feature selection, we can gain deeper insight into the underlying processes which led to variation in the data. Automated feature selection through deep learning networks have also been explored in healthcare literature. Despite their ability to select relevant techniques and features rapidly, they can limit comprehension of the phenomenon being classified. Additionally, they rely heavily on large amounts of data which may not be common in various medical datasets. Once relevant features have been identified from the data, machine learning models can be trained and evaluated. There are a myriad of feature selection techniques and machine learning models which have been used in various biomedical applications.

A technical advancement provided here is feature selection techniques and supervised machine learning models explored for detection of autonomic dysreflexia (AD). While previous approaches rely entirely on the pre-determined patterns in blood pressure measured by a telemetry system to detect the onset of AD event induced by a trigger, the system and methods described herein may apply a multimodal approach to detect the onset of AD. One technical advancement in particular is that the multi-modal approach may use machine learning to automate the process of detecting AD during onset using non-symptom-based approaches.

Provided is a non-invasive, multi-parametric approach to detect AD using the most efficient machine learning methods and feature selection techniques. Described in this section is feature extraction and selection procedures useful to develop an efficient machine learning model which can characterize the onset of AD. These feature selection techniques can also be applied in a variety of medical applications which do not have large datasets due to the relatively small population of persons with this condition

[symbol]

### Material and Approach

Dataset Preparation—For experimental purposes Sensor data was collected from 19 male Sprague Dawley rats. All animals were between 3-5 months of age and weighed 450-600 gms prior to spinal cord injury. These rats were given a spinal cord injury at the T2/T3 level and AD was induced through colorectal distension[23]. The experiments were performed in accordance with the international directions for the protection of animals used for scientific purposes and the protocol was approved by the Purdue University IACUC.

Sensors—Time-series data were collected from wearable ECG, skin nerve activity (skNA), blood pressure (BP), and skin temperature sensors from a restrained animal while it was awake. skNA allows non-invasive measurement of stellate ganglion nerve activity which provides sympathetic innervation to the heart, and has been validated in humans, rat and dog models. ECG and skNA were measured through gel-based electrodes placed in a Lead I configuration at the level of the right and left third ribs, with the electrode placed at the right leg serving as a reference electrode. The electrodes were connected to the Power Lab 26T bio-amplifier (AD Instruments, USA) and digitized with a sampling rate of 10 kHz and a recording bandwidth of 10 Hz-3 kHz.

Blood pressure (BP) was measured through a CODA 6-Channel High Throughput Non-Invasive Blood Pressure system (Kent Scientific, USA) [27]. The Coda system provides measurements of the systolic (SBP), diastolic (DBP) and mean (MAP) blood pressure from the tail of the animal. The BP values were measured two times a minute. The blood pressure system comprises an occlusion cuff placed at the base of the tail and a volume-pressure recording (VPR) cuff which is placed 2 inches from the base of the rat's tail.

Variations in the sampling rate were adjusted post-processing through timestamp matching. A 20 mmHg increase in systolic blood pressure when colorectal distension was induced was used as a gold standard to label the data collected from certain timestamps as either AD or non-AD datapoints.

Signal Processing—The data from the sensors was processed using filters to remove artifacts such as motion and other high-frequency noise. The ECG signal was processed using a 60 Hz notch filter to remove power line interference, and a seventh order Butterworth band-pass filter between 0.01-30 Hz to remove movement artefacts and other high frequency noise. Smoothing is often useful to suppress noise or interference on a signal and was done by using a moving average filter on the signal skNA is derived from the ECG signal using a band-pass filter between 500-1000 Hz.

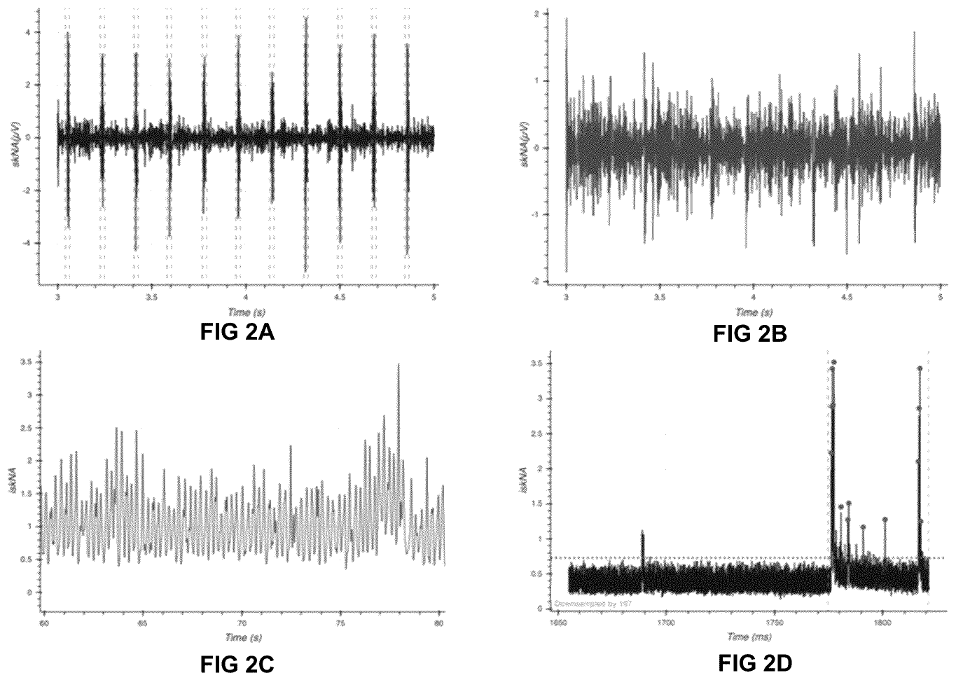

FIG. 2A-D illustrates plots of signals derived from an ECG signal via signal processing. FIG. 2A illustrates an example of a raw skNA signal with QRS interferences. FIG. 2B illustrates an example of a median filtered skNA signal without QRS interference. FIG. 2C illustrates an example of rectified and integrated skNA (iskNA). FIG. 2D illustrates an example of mean baseline value of non-bursting events (pink dotted horizontal line) and “burst” activity during sympathetic activation event (vertical dashed line) indicated by red dots.

The skNA signal contained interferences from QRS intervals (FIG. 2A). These QRS intervals were isolated through the Pan-Tompkins algorithm and smoothed using a median filter remove the interference (FIG. 2B). The signal was then rectified and integrated (iskNA) over a 100 ms window (FIG. 2C). Non-bursting baseline values of iskNA during rest were used to determine bursts in nerve activity. The mean of non-bursting iskNA plus 3 standard deviations (SD) were used as a threshold amplitude for determining bursting activity (FIG. 2D).

Feature Extraction—A fixed, sliding, non-overlapping window of 15 seconds was used to extract thirty-six relevant features. The detection of the QRS complexes and the R-peaks provide the fundamentals for almost all automated ECG analytics [32]. The Pan-Tompkins algorithm was used to extract the RR peaks as well as the QRS segments of each beat of the filtered ECG signal. To ensure detection accuracy, the derived RR peaks are further processed to ensure the minimum difference between two successive peaks is between 100-500 ms (200 bpm<HR<600 bpm) to generate the normal to normal (NN) intervals[33]. The heart rate and medianNN are calculated from the NN intervals. The PR interval, QR interval, QT interval, ST interval, PR segment and the ST segment which provide additional information about the cardiac condition were also extracted[34].

Heart rate variability (HRV) measures were also calculated from each window. These include the standard deviation of NN beat intervals (SDNN), covariance of NN intervals (covNN), the square root of the mean of the squares of the successive differences between adjacent NNs (RMSSD), and the proportion of the number of successive NN intervals which differ by more than 5 ms (NN5) as well as the percentage of NN5 (pNN5). The spectral power for HRV was analyzed on the windowed ECG segments. The total power (TP), very-low-frequency (VLF; 0.003-0.04 Hz), low-frequency (LF; 0.04-0.15 Hz), high-frequency (HF; 0.15-0.4 Hz) components were extracted from an FFT performed on the ECG signal. The peak amplitudes in VLF, LF, and HF components as well as the areas under these components were calculated. Additionally, the LF/HF ratio was also calculated.

The number of bursts, duration of bursts, Area under curve of the bursts were extracted from the iskNA. In addition, the average value of skNA and iskNA were extracted from each window. In addition, FFT performed on the skNA signal allowed extraction of the low, high and very high frequency bands of the sympathetic nerve activity.

After the features were extracted, they were normalized using a min-max scaler. For each feature value, we computed the z-score, that is the number of standard deviations the value was from its mean. Observations with a z-score greater than 3 were considered outliers and removed. Observations containing missing values, though rare, were discarded.

### Feature Selection

For classification and regression tasks, it is often useful to remove features which do not help model accuracy. The removal of extraneous variables tends to lower variance in the predicted values and reduces the likelihood of overfitting. Moreover, determining which features are useful in prediction can help point towards underlying mechanisms of the given problem, from which domain experts can work to develop new hypotheses. Below, discussed the approaches used for selecting useful features.

TABLE 1
Features extracted from the different sensors

| | Features | |
|---|---|---|
| Signal | Temporal: | Spectral: |
| ECG | 1. NN intervals | 9. Power of low frequency band |
| | 2. Heart rate | (0.01-0.75 Hz) − LF_{pow} |
| | 3. QRS interval | 10. Power of high frequency band |
| | 4. PR interval | (0.75-2.5 Hz) − HF_{pow} |
| | 5. MedianNN | 11. LFpow/HF pow |
| | 6. Number of NN | 12. Area under low frequency bands |
| | intervals <5 ms | (ALF) |
| | (nn5) | 13. Area under high frequency bands |
| | 7. Percentage of | (AHF) |
| | nn5 (pnn5) | 14. ALF/AHF ratio |
| | 8. covNN | |
| skNA | 15. Average | 21. Power of low frequency band |
| | skNA | (0-2.5 Hz) − LF_{pow} |
| | 16. Average | 22. Power of high frequency band |
| | iskNA | (2.5-5 Hz) − HF_{pow} |
| | 17. Area under | 23. Power of very high frequency |
| | Curve (AUC) | band (5-10 Hz) − VHF_{pow} |
| | skNA | 24. LF_{pow}/HF_{pow} |
| | 18. Number of | 25. Area under LF band (ALF) |
| | bursts | 26. Area under HF band (AHF) |
| | 19. Duration of | 27. Area under VHF band (AVHF) |
| | bursts | 28. ALF/AHF ratio |
| | 20. | AUC bursts |
| Skin | 29. | Δtemperature |
| Temperature | 30. | Mean temperature |
| | 31. | Median temperature |
| Blood | 32. | ΔSBP |
| Pressure | 33. | ΔDBP |
| | 34. | Mean SBP |
| | 35. | Mean DBP |
| | 36. | Mean arterial pressure (MAP) |

Univariate Filter Methods—Univariate feature selection allows the examination of each feature individually to measure its ability to determine the response variable. This often involves the computation of measures of association.

We computed a p-value through hypothesis testing (Student's t-test) and removed any features which did not meet a specific threshold (p<0.05). A chi-squared test was used to determine which features most closely resulted in changes in the features of the predictor.

We also used Pearson correlation-based feature selection wherein highly correlated features were removed. We removed predictors which are highly correlated (R²>0.7) with other predictors. While this approach is simple and can be reasonably effective, features which show higher-order or multivariate relationships with the response variable (but which individually do not show strong patterns) may unwittingly be discarded.

Best Subset Selection and Stepwise Search—Commonly used best subset regression techniques involve fitting and comparing 2^{P} possible models, wherein p is the number of features. However, this technique is often impractical for all but the smallest number of total features. In our case, with 30 features, 1 billion potential models need to be fit to determine the ones which lead to the best performance metrics. We used an iterative, stepwise, “greedy” search approach wherein a full model is initially built, and features are either successively added or removed from the dataset. We performed ‘recursive feature elimination’ starts by fitting a full model (containing all available features), and computes ‘feature importance’ values for each feature (e.g., for logistic regression, one could use the p-value from the Wald-tests for the coefficient parameters). We also used the inherent abilities of the decision tree to calculate a feature importance score from the Gini coefficient. Features whose feature importance does not meet a specified threshold were discarded. The procedure was then repeated, recursively, until all remaining features meet the threshold criteria, or until a target model dimension is achieved.

Recursive Feature Elimination—A recursive feature elimination (RFE) algorithm was used for feature selection. The RFE algorithm method attempts to find the best subset of size σ (σ<N) through a greedy backward selection. It chooses the σ features which lead to the largest margin of class separation by the logistic regression classifier. It iterates in a greedy fashion through the removal of input dimensions/features to decrease the margin of separation between the classes until only σ input dimensions remain. A binary logistic regression model was used for classification to identify the impact of the different features in predicting the onset of AD.

### Machine Learning Models

Eleven different classifiers were compared for the initial exploration of performance. These include K-Nearest Neighbor (KNN), linear and logistic regression, support vector machines (SVM) with linear and RBF kernels, Naïve Bayes, Quadratic Discriminant Analysis, ensemble methods such as random forest and Adaboost models, and neural networks (multilayer perceptron).

In order to train our machine learning models, we split the data into three stratified sets—the training set (70%), the test set (15%) and the validation set (15%). 10-fold cross-validation (CV) was used to create variations of the training, test and validation sets in order to reduce overfitting. The models were trained on the complete dataset as well as the reduced dataset developed from the feature selection methods.

### Performance Measures

We measured performance through a confusion matrix (Table 2). To determine the best performing algorithm, we used metrics of accuracy, sensitivity (true positive rate), specificity (true negative rate), and AUC-ROC score to evaluate the performance of the different models developed using the different feature selection techniques. Through the ROC curves, we were able to screen for the different types of errors which arise in many biomedical scenarios.

TABLE 2
Representation of the confusion matrix for
AD detection and metrics determination.

| | Predicted AD | Predicted Non-AD |
|---|---|---|
| Actual AD | True Positive (TP) | False Negative (FN) |
| Actual Non-AD | False Positive (FP) | True Negative (TN) |

### Results

Through the aforementioned feature selection approaches, we identified five relevant features which best characterized the onset of AD. These five features include medianNN, average iskNA, number of bursts, which are representative of sympathetic activity and RMSSD, pNN5 which are representative of vagal activity. These five features enabled a deeper insight into the biological processes involved in the resulting symptoms of AD. However, there is no ‘best’ feature selection procedure, as the choice of selection procedure highly depends on details of the problem at hand: the number of features, the availability of feature importance's, and the computational resources required by the model fitting procedure. The techniques presented in this paper provide a template which can be modified to suit the needs of other small dataset related projects. Thus, additional or alternative features to the 5 features may be used.

There is an observed overlap when visualizing AD and non-AD responses on a bivariate plot. However, the differences in the different distributions suggest the ability for discernment between the presence and absence of AD through the five features. These formed the basis of the separation between the two classes (AD and non-AD).

The reduced subset of features enabled us to develop and compare the eleven different models (Table 3). The best performing machine learning model developed using the reduced feature subset was a five-layer neural network (multi-layer perceptron) which had high accuracy (93.4%), sensitivity (93.5%) and specificity (93.3%). There was a notable increase in performance of the neural network when trained on the reduced feature subset when compared to the dataset without any feature selection

### Discussion

Feature selection performs a reduction in the complexity of a dataset to enable the development of reliable machine learning models [35]. Through better feature selection, it is possible to develop models which use physiological and healthcare data as an invaluable data source to assist in disease detection, rehabilitation and treatment[36]. In this paper, we compared different feature selection methods and machine learning models which enabled us to characterize the onset of AD with high-performance metrics.

These techniques can be used in different capacities to enable the development of machine learning models which are explainable, relevant and most importantly, perform well with clinically relevant physiological data. Machine learning models can enable early mitigation of AD leading to a reduction in related complications and mortality in individuals with SCI.

Relevance of Feature selection using small physiological datasets—With an increase in availability of wearable sensing technologies, such as the Apple™ Watch, Fitbit™, there is an increasing amount of healthcare data that can be collected and made available to clinicians and others in the field of healthcare. This leads to a voluminous number of features, which can be extracted allowing a richer understanding of the biological processes involved in various disease states instead of being limited in collecting data in controlled settings. Unfortunately, this development of increasingly complex datasets which have a great deal of inter-related features serves to complicate straightforward discrimination of results necessitating the development of machine learning models. There is a need to provide efficient, parallel data processing techniques to develop efficient machine learning models, which is made possible through feature selection. Feature selection is particularly important when making predictions regarding the outcomes or onset of diseases.

Through the feature selection approaches presented in this paper, we were able to narrow our feature subset. The selection of five features rather than thirty-six enabled a sharper focus on relevant changes occurring in the physiology due to the onset of AD. However, there is no ‘best’ feature selection procedure, as the choice of selection procedure highly depends on details of the problem at hand: the number of features, the availability of feature importances, and the computational resources required by the model fitting procedure. The techniques presented in this paper provide a template which can be modified to suit the needs of other small dataset related projects.

Relevance of neural network performance—From experimentation, a feedforward neural network arguably showed the strongest overall performance, including the highest accuracy and AUC score among the models tested (See Table 3 below). A Gaussian Process model performed similarly, but with slightly lower accuracy and AUC score. These results indicate that there are likely important nonlinear relationships within our data, as neural networks and Gaussian processes are two of the more flexible supervised learning models. In our case, the neural network contained a total of ˜2000 parameters (and Gaussian processes are non-parametric). It is not too surprising that these two models performed similarly, as it is known that neural networks, in a sense, approximate Gaussian processes. However, may types of supervised machine learning models may be applied, depending on the implementation.

TABLE 3
Performance Metrics for the Different
Classifiers with the AD Dataset

| | Accuracy | Sensitivity | Specificity | AUC- |
|---|---|---|---|---|
| Name | (%) | (%) | (%) | ROC |
| Neural Network (without | 72.2 | 70.1 | 76.7 | 0.74 |
| feature selection) | | | | |
| Neural Network | 93.4 | 93.5 | 93.3 | 0.93 |
| Adaboost | 79.3 | 79.3 | 79.2 | 0.78 |
| Decision Tree | 86.1 | 83.3 | 89.5 | 0.86 |
| Gaussian Process | 91.7 | 88.9 | 94.4 | 0.92 |
| K Nearest Neighbor | 86.5 | 83.3 | 89.5 | 0.86 |
| Linear SVM | 62.2 | 30.1 | 86.7 | 0.61 |
| Logistic Regression | 87.4 | 84.3 | 82.5 | 0.87 |
| RBF SVM | 63.9 | 72.2 | 84.2 | 0.64 |
| Naïve Bayes | 88.9 | 94.4 | 83.3 | 0.89 |
| Random Forest | 63.9 | 72.2 | 84.2 | 0.64 |

A drawback of the more flexible models is that they tend to require relatively more data to achieve good performance. On the other hand, as the size of data grows, they tend to better detect subtle relationships that may exist. Consequently, as more data becomes available, further improvements in the performance of the neural network and Gaussian process models (as well as the other more flexible models) is projected.

We do note that although the more flexible models showed the strongest performance, two of the simpler models—logistic regression and quadratic discriminant analysis—showed reasonably strong performance as well. This suggests that while complex nonlinear relationships may exist within the data, much of the variation in the response is accounted for by first and second-order terms of the features. In a setting where the number of observations is relatively small, it may be more prudent to consider the simpler methods, as they tend to be relatively more stable (low variance), especially for smaller datasets.

Clinical Relevance in Autonomic Dysreflexia—Recognition and prevention of AD related signs and symptoms plays a critical role in avoiding escalation to more dire circumstances in clinical and non-clinical environments. Currently the standard approach for managing AD is to train persons with SCI to recognize their symptoms and to promptly alleviate the AD trigger, which can be difficult to identify and frequently requires the assistance of a caregiver. There is a need for a need for a sensitive yet noninvasive method of detecting the onset of AD, which can be adopted easily into clinical practice and for at home use.

The major findings of the multi-modal experimentation and examples described herein demonstrate that there are alternate techniques to determining the onset of AD through non-invasive wearable sensing techniques.

These could be complementary to current clinical tools. A non-invasive sensor system that can automatically detect the onset of AD, can improve independence and quality of life of individuals with an SCI. Additionally, such a detection system could allow individuals with more time to identify and eliminate the trigger before escalation to dangerous hypertensive levels.

### Examples and Experiments With Predicting AD Using SKNA

In some examples, a neural network model may be used to accurately detecting AD events using only the SKNA signal. For example, a DNN model was trained on labeled data sets and outperformed previous methods that utilized multiple physiological features, demonstrating that SKNA alone is sufficient for detecting AD-related changes in the autonomic nervous system. The model's performance was evaluated on 30% of unseen data, confirming its robustness and ability to generalize well.

The significance of each extracted feature in distinguishing AD events from non-AD events was explored. Each feature provides unique information, and our statistical metrics reveal distinct patterns between the two groups. Variance, Kurtosis, Root Mean Square (RMS), Waveform Length (WL), Zero Crossings (ZC), Slope Sign Change (SSC), Amplitude-Waveform Area (WA), and Crest Factor (CF) were among the key features analyzed, highlighting their potential impact on the classification of AD events. These results contribute to a deeper understanding of AD and pave the way for potential diagnostic advancements.

Feature Analysis—Various statistical metrics, including maximum, mean, minimum, and standard deviation, were calculated to understand the differences between the two groups. Our results revealed distinct patterns in the analyzed features between the AD and Non-AD groups. Firstly, in terms of Variance, the AD group exhibited higher values (maximum: 0.001528, mean: 0.000463) compared to the Non-AD group (maximum: 0.0008, mean: 0.000339), indicating increased variability in the skin nerve activity signals among individuals with AD. Similarly, the Kurtosis values were higher in the AD group (maximum: 54.71572, mean: 25.625879) compared to the Non-AD group (maximum: 39.480322, mean: 22.41112), suggesting more pronounced peakiness and heavier tails in the distribution of the nerve activity signals for individuals with AD. Moving on to RMS, the AD group had higher values (maximum: 0.087063, mean: 0.053605) compared to the Non-AD group (maximum: 0.084781, mean: 0.034548), indicating larger overall amplitudes in the skin nerve activity signals among individuals with AD. Similarly, the WL values were higher in the AD group (maximum: 1229.583535, mean: 874.294221) compared to the Non-AD group (maximum: 811.722025, mean: 657.936163), suggesting more complex and intricate waveforms in the nerve activity signals of individuals with AD. Furthermore, the ZC values were higher in the AD group (maximum: 23919.58824, mean: 17788.39685) compared to the Non-AD group (maximum: 17730.47059, mean: 15530.09685), indicating a higher frequency of changes in signal polarity in the nerve activity signals for individuals with AD. Similarly, the SSC values were slightly higher in the AD group (maximum: 25649.76471, mean: 22915.20846) compared to the Non-AD group (maximum: 25129.41177, mean: 22552.04408), suggesting more frequent changes in signal slope in the nerve activity signals for individuals with AD.

Additionally, the WA values were higher in the AD group (maximum: 32596.2353, mean: 26062.50403) compared to the Non-AD group (maximum: 30388.76471, mean: 24471.86757), indicating larger amplitudes in the nerve activity signals for individuals with AD. Lastly, the CF values were higher in the AD group (maximum: 16.170041, mean: 10.15679) compared to the Non-AD group (maximum: 14.36859, mean: 8.82605), suggesting sharper peaks in the waveform of the skin nerve activity signals for individuals with AD.

Classification Performance—First and foremost, the model exhibited exceptional accuracy in identifying AD cases, achieving an average accuracy of 93.9% (±2.5%). This indicates that the model was highly proficient in differentiating between AD and non-AD cases, with a relatively low error rate. These findings suggest that the DNN model holds significant promise as a robust tool for AD detection in rats induced by CRD. To further evaluate the model's performance, we employed the F1-score, a metric that balances precision and recall. The average F1-score obtained was 0.944 (±0.018), indicating a commendable balance between accurately identifying true positive AD cases and minimizing false positives and negatives. This high F1-score underscores the model's ability to balance capturing AD cases and avoiding misclassification. Examining the false negatives, we found an average value of 4.8% (±1.6%). This implies that the model incorrectly classified a small proportion of non-AD cases as AD. While this rate is relatively low, it does highlight the importance of further improving the model's sensitivity to ensure the accurate identification of all AD cases. Nevertheless, it is worth noting that the model consistently achieved an impressive average true positive rate of 95.2% (±2.6%), demonstrating its effectiveness in capturing the most true positive AD cases. Additionally, the model demonstrated high precision and recall, with average values of 95.2% (±2.1%) for both metrics. This indicates the model's ability to achieve high precision in correctly identifying AD cases and high recall in capturing most of the AD cases among all positive predictions. These results further validate the model's efficacy in accurately detecting AD cases induced by CRD in rats. The observed standard deviations in accuracy, F1-score, false negatives, true positive rate, precision, and recall reflect the variability in model performance across different rats. They also underscore the importance of considering individual variations in the interpretation of the results. However, it is noteworthy that the relatively small standard deviations suggest consistent and stable model performance. This stability is indicative of the model's robustness and reliability, as its accuracy appears to be less influenced by rat-specific factors.

### Discussion

To ensure the reliability and validity of our results, we meticulously reviewed our data and found that three rats had passed away prematurely prior to the completion of the experiment. Consequently, we have appropriately excluded these rats from our analysis to ensure that our findings are robust and accurately reflect the intended population. The DNN model demonstrated high precision and recall values for the majority of rats, indicating a low rate of false positives and a high detection of true positives. The F1 scores further supported the model's overall efficiency in identifying true positives. Additionally, the sensitivity and specificity values demonstrated a high level of accuracy in detecting both true positives and true negatives. The model showed promising results, and the false negative rate was relatively low for most rats, suggesting that the model could be a solution for

[symbol]

AD detection. The lowest accuracy achieved was 92.2%, indicating its ability to correctly identify the majority of AD cases. This result underscores the model's efficacy in accurately classifying AD cases, even in challenging circumstances. Furthermore, the lowest F1 score achieved was 93.6%, representing a remarkable balance between precision and recall. This score highlights the rat parameter's ability to achieve a high level of precision in correctly identifying AD cases while effectively capturing a significant proportion of true positive AD cases among all positive predictions. We employed scatter plots to analyze the classification performance of AD and baseline episodes in rats subjected to CRD over a duration of 1 minute. However, we encountered a challenge as certain features of AD and baseline episodes exhibited overlapping patterns, which hindered the use of linear classification methods.

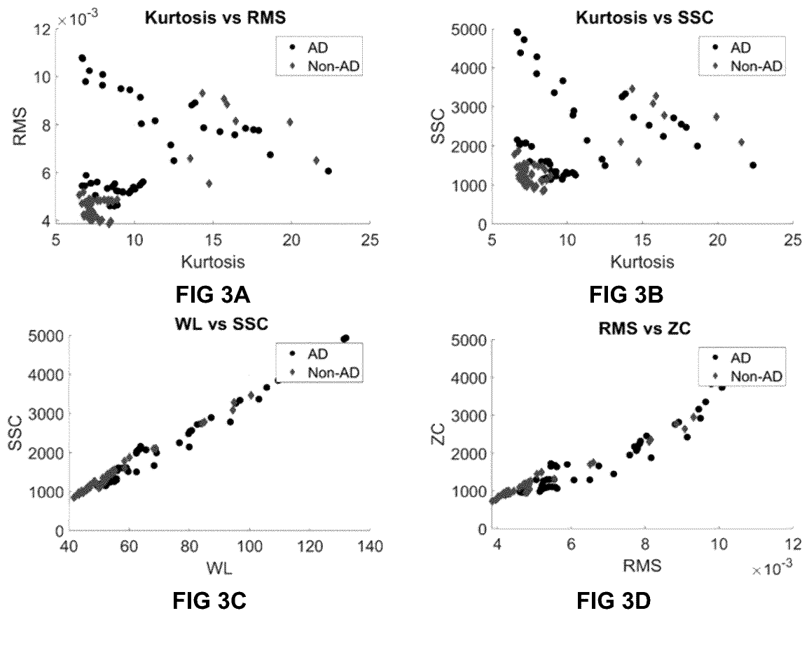

FIG. 3A-D illustrates an example of correlation of various SKNA features utilized for the classification of AD and non-AD events, along with the feature distribution. The four scatter plots demonstrate the relationship between the features and their distribution for classification. The plots Kurtosis vs RMS (FIG. 3A), Kurtosis vs SSC (FIG. 3B), WL vs SSC (FIG. 3C), and RMS vs ZC (FIG. 3D), highlight the effectiveness of the SKNA features in distinguishing between AD and non-AD events. These findings support using the proposed system as a reliable tool for detecting and monitoring AD in individuals with spinal cord injuries.

We observed that AD episodes displayed distinct characteristics in terms of SKNA activity distribution, neuron spiking, and signal amplitude or power. Notably, AD episodes exhibited a pronounced increase in these aspects compared to baseline episodes. This was evident in scatter plots as shown in FIG. 3A-B, such as Kurtosis vs. RMS or RMS vs. SSC, where a substantial number of observations clustered in regions associated with heightened neuronal activity. The clustering of non-Ad events suggests that they tend to occur near each other, while AD events are scattered and spread out widely. The SSC analysis indicates that when an AD event occurs, there is a higher number of neuronal firings originating from the stellate ganglion. This observation is consistent with the ZC vs RMS plot, where the occurrence of a burst leads to an increased number of ZCs. Examining the shape or kurtosis feature reveals an interesting pattern. During non-AD events, the kurtosis values are distributed closely together. However, when an AD event occurs, the kurtosis values are spread out further and are not tightly distributed. This suggests that the shape and duration of the bursts can vary significantly during AD events. In summary, the observed patterns indicate that non-Ad events tend to cluster closely together, while AD events are more scattered and widespread. The analysis of SSC and ZC vs RMS plots suggests that the stellate ganglion is more active during AD events. Additionally, the kurtosis values show that the shape and duration of bursts can vary greatly during AD events compared to non-AD events. Further, we were able to capture the distinct characteristics of AD episodes that would have been challenging to discern using linear classification methods alone. This highlights the significance of considering multiple parameters and non-linear approaches in the analysis of complex physiological phenomena like AD.

### Materials and Approach

Data Acquisition—Fifteen male Sprague Dawley rats were purchased from Envigo (Indianapolis, IN) and implanted with a single pressure and biopotential implant (HD-S11 Implant, DSI International, USA) to record blood pressure and ECG in real-time with a sampling rate of 1 kHz. Male rats were selected exclusively for the study due to the high incidence of SCI in men, who account for around 80% of all cases and tend to sustain injuries at an earlier age. Additionally, non-invasive equipment was used to record SKNA and ECG at a sampling rate of 10 kHz21. The animals were housed individually in a Plexiglas cage with straw bedding and were provided with ad libitum access to food and water. After a four-week acclimation period to the experimental setup and sensors, including electrodes, a restraining jacket, and a plastic holder with air holes (HLD-RL model, Kent Scientific, USA), each rat underwent a dorsal laminectomy followed by a spinal cord crush with specially designed forceps. This resulted in the loss of motor function in both hind legs of the rats, and a recovery period of five days was allowed before experimentation.

For each experiment, ECG electrodes were placed on the rats in the Lead I configuration, and the rats were placed in a restraining jacket that was subsequently placed in a restraining tube. The onset of AD was induced by CRD performed on the rats. A balloon catheter was inserted into the rectum, and 20 minutes of baseline data were recorded. Subsequently, the catheter was inflated for one minute every ten minutes for a total of 30 minutes. The protocol used for this study was approved by the Purdue University Animal Care and Use Committee. Overall, this experimental setup allowed for the simultaneous recording of multiple physiological parameters, including blood pressure, ECG, and skin nerve activity, providing a comprehensive data set for the analysis of AD in rats. This project has received approval from the Purdue Animal Care and Use Committee (PACUC) with the assigned approval number being 1810001814.

AD and Non-AD segmentation from SKNA recording—An implanted sensor simultaneously recorded arterial BP as the gold standard for determining AD events before, during, and after CRD procedures. AD events were identified based on the presence of systolic blood pressure exceeding 15 mmHg accompanied by bradycardia.

For developing a robust machine learning model, the SKNA signal was segmented between AD and non-AD events. Each AD event lasting one minute was segmented and paired with a corresponding non-AD event from the baseline of the same duration. Non-AD events were chosen from the baseline recording, which may include some skin SKNA spikes due to hypertension or other factors in the rat model. Randomly selected non-AD events from the baseline were used for training to ensure that the machine learning model was capable of handling real-world factors such as noise and motion artifacts. The baseline recording showed a single spike, while several SKNA bursts were observed during the CRD.

AD and Non-AD segmentation from SKNA recording—An implanted sensor simultaneously recorded arterial BP as the gold standard for determining AD events before, during, and after CRD procedures. AD events were identified based on the presence of systolic blood pressure exceeding 15 mmHg accompanied by bradycardia.

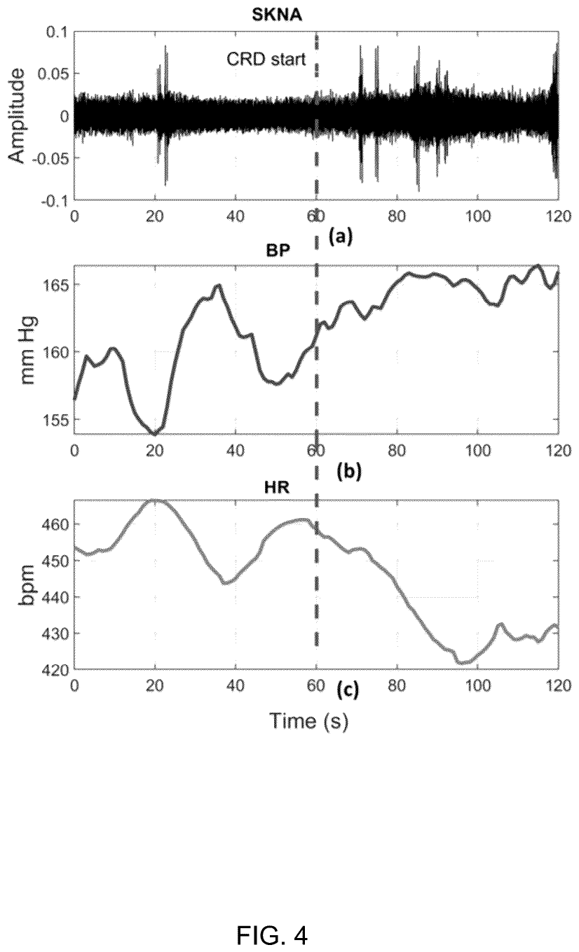

FIG. 4 illustrates an AD event that occurred during the performance of CRD. a) A SKNA signal during baseline and during CRD (after blue dotted line) with the induction of an AD event, (b) Blood pressure measurement before and during CRD, (c) heart rate (HR) before and during the CRD, with an immediate decrease observed after the rise in blood pressure

For developing a robust machine learning model, the SKNA signal was segmented between AD and non-AD events. Each AD event lasting one minute was segmented and paired with a corresponding non-AD event from the baseline of the same duration. Non-AD events were chosen from the baseline recording, which may include some skin SKNA spikes due to hypertension or other factors in the rat model. Randomly selected non-AD events from the baseline were used for training to ensure that the machine learning model was capable of handling real-world factors such as noise and motion artifacts. The baseline recording showed a single spike, while several SKNA bursts were observed during the CRD.

TABLE 4
Feature Descriptions and Formulas

| Feature | Description | Formula |
|---|---|---|
| Variance | Measures the spread of the signal around its mean value, providing information about the signal's | $Variance = \frac{1}{N} \sum {( x - μ )}^{2}$ |
| | variability. | |
| Kurtosis | Measures the peakiness of the distribution of the signal's amplitude, providing information about the | $Kurtosis = \frac{1}{N} \sum {( \frac{x - μ}{σ} )}^{4} - 3$ |
| | signal's distribution. | |
| RMS | Calculates the root mean square of the signal, representing the signal's power and energy. | $RMS = \sqrt{\frac{1}{N} \sum ( {χ}^{2} )}$ |
| Wave | Provides information about the signal's shape and | WL = Σ\|x(i) − x(i − 1)\| |
| Length | frequency content by measuring the cumulative | |
| | length of the signal's waveform. | |
| Zero Crossing | Counts the number of times the signal crosses the zero axis, providing information about the signal's | $ZC = \frac{1}{N} \sum \vert sign ( x ( i ) ) - sign ( x ( i - 1 ) ) \vert$ |
| | frequency. | |
| Slope Sign | Counts the number of times the slope of the signal | SSC = Σ(sign(diff(x))/ = 0) |
| Change | changes sign, providing information about the signal's | |
| | rate of change. | |
| Willison | Calculates the weighted sum of the absolute | WAMP = Σ(\|x(i) − x(i − 1)\| > T) |
| Amplitude | differences between adjacent samples that exceed a | |
| | threshold value, providing information about the | |
| | signal's amplitude changes over time. | |
| Crest | Measures the peak-to-average ratio of the signal, | Crest Factor = RMS Value Peak |
| Factor | providing information about the signal's dynamic range. | Value |

Signal pre-processing and feature extraction—The signal processing approach involved bandpass filtering the time series data between 500-1000 Hz using an 8th-order Butterworth filter to remove any unwanted noise or artifacts that could negatively impact the feature extraction process. The resulting filtered signal was then normalized to the range −1 to 1, which is a common and effective pre-processing step in the signal analysis27.

In a previous study, five potential SKNA signal biomarkers for AD detection were identified, including medianNN, average iSKNA, number of bursts, RMSSD, and pNN516. However, we discovered that the RMSSD and pNN5 features were delayed in detecting AD, and identifying bursts in the iSKNA signal was difficult due to varying thresholds based on amplitude28. To overcome these limitations, we decided to focus exclusively on the SKNA signal and selected features that were not dependent on specific thresholds. The selected features for the proposed model, such as Variance, Kurtosis, RMS, WL, ZC, SSC, WA, and CF, capture different aspects of the signal's characteristics. They provide information about the signal's variability, distribution, power, waveform, frequency content, rate of change, amplitude changes, and dynamic range as defined in Table 4. The combination of these features offers a diverse set of information about the skin nerve activity signals. By capturing different aspects of the signals, they provide a comprehensive representation that helps the DNN model learn relevant patterns and discriminative features. We used an overlapping window technique with a window length of 5 seconds and a 0.5-second overlap to derive the features from the SKNA signals. This ensured the accuracy and robustness of the extracted features that truly represented the desired behavior in the signals.

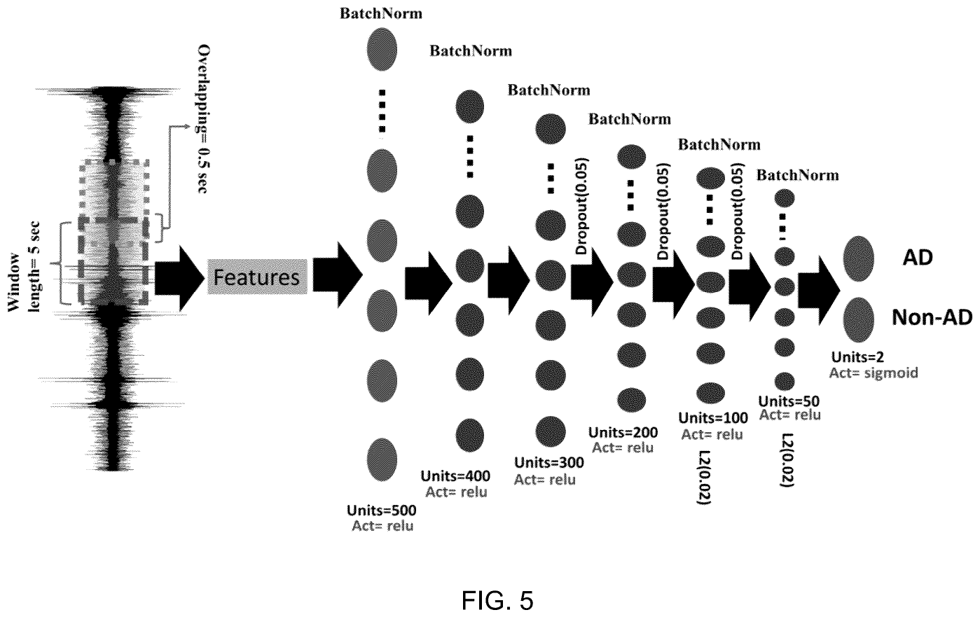

FIG. 5 illustrates an example of a neural network architecture for detecting AD and Non-AD events. The various layers and connections between them enabled the model to classify the input data.

Classification Algorithm and Analysis Matrices—DNNs have been found effective in diagnosing diseases by classifying biological signals. The proposed DNN architecture includes six dense layers using varied numbers of neurons and regularization techniques as shown in FIG. 5. The first dense layer has 500 neurons and accepts the training features' input shape. Batch normalization was applied after each dense layer to normalize the input for the following layer. The second dense layer had 400 neurons, followed by the third dense layer with 300 neurons. The fourth and fifth dense layers have 200 and 100 neurons, respectively. The final dense layer had 50 neurons, applying L2 regularization with a parameter of 0.02 and using the sigmoid activation function for binary classification. To avoid overfitting, dropout regularization with a 0.05 rate was applied after the third and fourth dense layers. The model was compiled with binary cross-entropy loss and stochastic gradient descent optimizer. The model was trained with 500 epochs using a batch size of 32 for training data, with validation data provided during training. The model's weights were saved with the best validation accuracy using a checkpoint callback. By utilizing DNN techniques, this approach provided an effective tool for automated AD diagnosis from SKNA signals.

To evaluate the performance of the proposed model, commonly used metrics such as True Positives (TP), False Positives (FP), True Negatives (TN), False Negatives (FN), Precision, Recall, F1-score, Sensitivity, and Specificity were used. TP represents correctly classified positive samples, FP represents negative samples incorrectly classified as positive,

TN represents correctly classified negative samples, and FN represents positive samples incorrectly classified as negative. Precision is the fraction of true positives out of all samples classified as positive, while Recall is the fraction of true positives out of all actual positive samples. The F1-score is the harmonic mean of precision and recall. Sensitivity represents the true positive rate, or the fraction of actual positive samples that are correctly classified as positive, while Specificity represents the true negative rate or the fraction of actual negative samples that are correctly classified as negative. These metrics will help in assessing the model's ability to distinguish between AD and non-AD events

This paper presents an AI-powered solution for the detection and monitoring of AD in individuals with SCI using non-invasive sensors to measure changes in sympathetic activity. The proposed system overcomes the limitations of current AD detection methods and blood pressure monitoring systems by utilizing the skin nerve activity signal and deep neural network architecture with high precision, high recall, and low false-negative rates. The system's robustness and ability to generalize well were confirmed by evaluating its performance on naïve data. This research represents a significant advancement towards noninvasive, real-time monitoring of AD that may be translatable to humans with SCI. The potential for early detection and timely intervention using this AI-powered solution before AD symptoms dangerously escalate could significantly enhance patient outcomes and improve the management of AD. The development of wearable technology for AD detection may enable individuals with SCI to continuously monitor their condition wherever they are promoting greater independence and quality of life for individuals with high-level SCI.

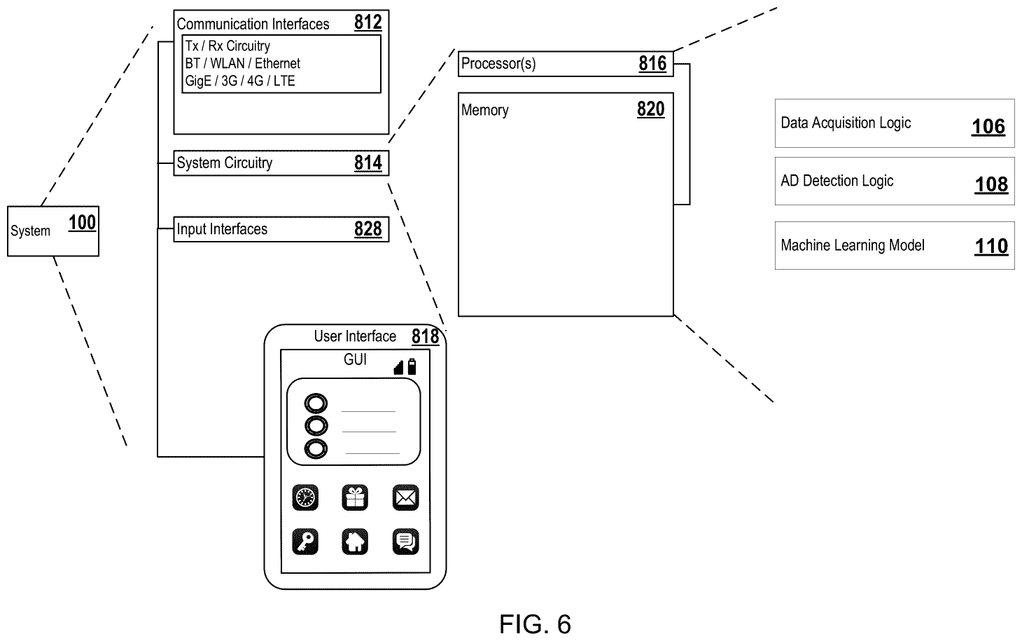

FIG. 6 illustrates a second example of the system. The system 100 may include communication interfaces 812, input interfaces 828 and/or system circuitry 814. The system circuitry 814 may include a processor 816 or multiple processors. Alternatively or in addition, the system circuitry 814 may include memory 820.

The processor 816 may be in communication with the memory 820. In some examples, the processor 816 may also be in communication with additional elements, such as the communication interfaces 812, the input interfaces 828, and/or the user interface 818. Examples of the processor 816 may include a general processor, a central processing unit, logical CPUs/arrays, a microcontroller, a server, an application specific integrated circuit (ASIC), a digital signal processor, a field programmable gate array (FPGA), and/or a digital circuit, analog circuit, or some combination thereof.

The processor 816 may be one or more devices operable to execute logic. The logic may include computer executable instructions or computer code stored in the memory 820 or in other memory that when executed by the processor 816, cause the processor 816 to perform the of the data acquisition logic, AD detection logic, machine learning model, and/or the system 100. The computer code may include instructions executable with the processor 816.

The memory 820 may be any device for storing and retrieving data or any combination thereof. The memory 820 may include non-volatile and/or volatile memory, such as a random access memory (RAM), a read-only memory (ROM), an erasable programmable read-only memory (EPROM), or flash memory. Alternatively or in addition, the memory 820 may include an optical, magnetic (hard-drive), solid-state drive or any other form of data storage device. The memory 820 may include at least one of the data acquisition logic, AD detection logic, machine learning model, and/or the system 100. Alternatively or in addition, the memory may include any other component or sub-component of the system 100 described herein.

The user interface 818 may include any interface for displaying graphical information. The system circuitry 814 and/or the communications interface(s) 812 may communicate signals or commands to the user interface 818 that cause the user interface to display graphical information. Alternatively or in addition, the user interface 818 may be remote to the system 100 and the system circuitry 814 and/or communication interface(s) may communicate instructions, such as HTML, to the user interface to cause the user interface to display, compile, and/or render information content. In some examples, the content displayed by the user interface 818 may be interactive or responsive to user input. For example, the user interface 818 may communicate signals, messages, and/or information back to the communications interface 812 or system circuitry 814.

The system 100 may be implemented in many different ways. In some examples, the system 100 may be implemented with one or more logical components. For example, the logical components of the system 100 may be hardware or a combination of hardware and software. The logical components may include the data acquisition logic, AD detection logic, machine learning model, or any component or subcomponent of the system 100. In some examples, each logic component may include an application specific integrated circuit (ASIC), a Field Programmable Gate Array (FPGA), a digital logic circuit, an analog circuit, a combination of discrete circuits, gates, or any other type of hardware or combination thereof. Alternatively or in addition, each component may include memory hardware, such as a portion of the memory 820, for example, that comprises instructions executable with the processor 816 or other processor to implement one or more of the features of the logical components. When any one of the logical components includes the portion of the memory that comprises instructions executable with the processor 816, the component may or may not include the processor 816. In some examples, each logical component may just be the portion of the memory 820 or other physical memory that comprises instructions executable with the processor 816, or other processor(s), to implement the features of the corresponding component without the component including any other hardware. Because each component includes at least some hardware even when the included hardware comprises software, each component may be interchangeably referred to as a hardware component.

Some features are shown stored in a computer readable storage medium (for example, as logic implemented as computer executable instructions or as data structures in memory). All or part of the system and its logic and data structures may be stored on, distributed across, or read from one or more types of computer readable storage media. Examples of the computer readable storage medium may include a hard disk, a floppy disk, a CD-ROM, a flash drive, a cache, volatile memory, non-volatile memory, RAM, flash memory, or any other type of computer readable storage medium or storage media. The computer readable storage medium may include any type of non-transitory computer readable medium, such as a CD-ROM, a volatile memory, a non-volatile memory, ROM, RAM, or any other suitable storage device.

The processing capability of the system may be distributed among multiple entities, such as among multiple processors and memories, optionally including multiple distributed processing systems. Parameters, databases, and other data structures may be separately stored and managed, may be incorporated into a single memory or database, may be logically and physically organized in many different ways, and may implemented with different types of data structures such as linked lists, hash tables, or implicit storage mechanisms. Logic, such as programs or circuitry, may be combined or split among multiple programs, distributed across several memories and processors, and may be implemented in a library, such as a shared library (for example, a dynamic link library (DLL).

All of the discussion, regardless of the particular implementation described, is illustrative in nature, rather than limiting. For example, although selected aspects, features, or components of the implementations are depicted as being stored in memory(s), all or part of the system or systems may be stored on, distributed across, or read from other computer readable storage media, for example, secondary storage devices such as hard disks, flash memory drives, floppy disks, and CD-ROMs. Moreover, the various logical units, circuitry and screen display functionality is but one example of such functionality and any other configurations encompassing similar functionality are possible.

The respective logic, software or instructions for implementing the processes, methods and/or techniques discussed above may be provided on computer readable storage media. The functions, acts or tasks illustrated in the figures or described herein may be executed in response to one or more sets of logic or instructions stored in or on computer readable media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one example, the instructions are stored on a removable media device for reading by local or remote systems. In other examples, the logic or instructions are stored in a remote location for transfer through a computer network or over telephone lines. In yet other examples, the logic or instructions are stored within a given computer and/or central processing unit (“CPU”).

Furthermore, although specific components are described above, methods, systems, and articles of manufacture described herein may include additional, fewer, or different components. For example, a processor may be implemented as a microprocessor, microcontroller, application specific integrated circuit (ASIC), discrete logic, or a combination of other type of circuits or logic. Similarly, memories may be DRAM, SRAM, Flash or any other type of memory. Flags, data, databases, tables, entities, and other data structures may be separately stored and managed, may be incorporated into a single memory or database, may be distributed, or may be logically and physically organized in many different ways. The components may operate independently or be part of a same apparatus executing a same program or different programs. The components may be resident on separate hardware, such as separate removable circuit boards, or share common hardware, such as a same memory and processor for implementing instructions from the memory. Programs may be parts of a single program, separate programs, or distributed across several memories and processors.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

1. A method, comprising:
measuring skin nerve activity (skNA), galvanic skin response (GSR), heart rate, and skin temperature of a subject;
extracting, a plurality of features from the measurements, the features comprising medianNN, average iskNA, number of bursts, RMSSD, pNN5, or a combination thereof;
classifying, based on a machine learning model, the plurality of features to identify the onset of autonomic dysreflexia (AD) in the subject; and
outputting, in response to the onset of AD, a message indicative of the onset of AD.

2. The method of claim 1, wherein measuring skin nerve activity (skNA), galvanic skin response (GSR), heart rate, and skin temperature of a subject further comprises
receiving signals from a plurality of non-invasive sensors attached to a subject, the sensors comprising an ECG sensor, a GSR sensor, a heart rate monitor, and a skin temperature sensor.

3. The method of claim 1, wherein the machine learning model comprises a neural network.

4. The method of claim 4, wherein the neural network comprises a multilayer perceptron model or a convolutional neural network which can be trained using data collected.

5. The method of claim 1, wherein the machine learning model is trained based on a plurality of training data comprising labeled features derived from time series data from skin nerve activity (skNA) data, GSR data, and a skin temperature data.

6. The method of claim 1, wherein the skin nerve activity (skNA), galvanic skin response (GSR), heart rate, and skin temperature each have a sampling resolution less than 30 seconds.

7. The method of claim 1, wherein outputting, in response to the onset of AD, the message indicative of the onset of AD further comprises, storing the message in a memory, communicating the message over a network, causing the message to be displayed, or a combination thereof.

8. A method, comprising:
measuring skin nerve activity (SKNA) of a subject over a time window;
extracting, a plurality of features from the measured SKNA, the features comprising variance, kurtosis, root mean square (RMS), wave length, zero crossing, slope sign change, Willison amplitude, and crest factor;
classifying, based on a machine learning model, the plurality of features to identify the onset of autonomic dysreflexia (AD) in the subject; and
outputting, in response to the onset of AD, a message indicative of the onset of AD.

9. The method of claim 8, wherein classifying, based on a machine learning model, the plurality of features to identify the onset of autonomic dysreflexia (AD) in the subject further comprises:
supplying the features to a deep neural network.

10. The method of claim 9, wherein the deep neural network was previously trained to identify the onset of AD based on training features having a same type as the extracted features.

11. The method of claim 8, wherein in measuring skin nerve activity (SKNA) of a subject over a time window comprises:
receiving a signal from an ECG sensor attached to the subject.

12. The method of claim 8, wherein outputting, in response to the onset of AD, the message indicative of the onset of AD further comprises, storing the message in a memory, communicating the message over a network, causing the message to be displayed, or a combination thereof.

13. A system, comprising a hardware processor, the hardware processor configured to:
measure skin nerve activity (skNA), galvanic skin response (GSR), heart rate, and skin temperature of a subject;
extract, a plurality of features from the measurements, the features comprising medianNN, average iskNA, number of bursts, RMSSD, pNN5, or a combination thereof;
classify, based on a machine learning model, the plurality of features to identify the onset of autonomic dysreflexia (AD) in the subject; and
output, in response to the onset of AD, a message indicative of the onset of AD.

14. The system of claim 13, wherein to measure skin nerve activity (skNA), galvanic skin response (GSR), heart rate, and skin temperature of a subject, the hardware processor is further configured to:
receive signals from a plurality of non-invasive sensors attached to a subject, the sensors comprising an ECG sensor, a GSR sensor, a heart rate monitor, and a skin temperature sensor.

15. The system of claim 13, wherein the machine learning model comprises a neural network.

16. The system of claim 15, wherein the neural network comprises a multilayer perceptron model or a convolutional neural network which can be trained using data collected.

17. The system of claim 13, wherein the machine learning model is trained based on a plurality of training data comprising labeled features derived from time series data from skin nerve activity (skNA) data, GSR data, and a skin temperature data.

18. The system of claim 13, wherein the ECG, skin nerve activity (skNA), galvanic skin response (GSR), heart rate, and skin temperature each have a sampling resolution less than 30 seconds.

19. The system of claim 13, wherein to output, in response to the onset of AD, the message indicative of the onset of AD, the hardware processor is further configured to store the message in a memory, communicating the message over a network, causing the message to be displayed, or a combination thereof.
